(19) United States
(12) Patent Application Publication
(10) Pub. No.: US 2025/0152385 A1
(43) Pub. Date: May 15, 2025

(54) EXTERNAL FIXATION ASSEMBLY FOR POST-AMPUTATION PROSTHESIS

(71) Applicants: Dennis G. Haun, Fallston, MD (US); Christopher Bibbo, Reisterstown, MD (US)

(72) Inventors: Dennis G. Haun, Fallston, MD (US); Christopher Bibbo, Reisterstown, MD (US)

(21) Appl. No.: 18/947,114

(22) Filed: Nov. 14, 2024

Related U.S. Application Data
(60) Provisional application No. 63/548,436, filed on Nov. 14, 2023.

(51) Int. Cl. A61F 2/80; A61F 2/50; A61F 2/60

(52) U.S. Cl. CPC A61F 2/80; A61F 2002/5038; A61F 2002/5096; A61F 2002/607

(57) ABSTRACT

A fixation assembly for quick-connect adjustable attachment of a prosthetic leg to an external ring fixator for immediate post-amputee ambulation. The device is configured for use with an existing external fixation assembly attached to a femur or tibia, and an existing prosthetic leg and foot combination of choice. The assembly generally includes an articulating fixator adjustably-attached to a monolithic support block by locking-ball-joint struts, and quick-connect pins insertable through the fixator and into receptacles on the locking-ball-joint struts for releasable attachment. The monolithic support block is a unitary block configured as a mounting base for mounting the prosthetic leg/foot support to its underside, and upwardly configured for weight-bearing attachment of the locking-ball-joint struts. The quick-connect pins allow releasable mounting to the existing external ring fixator.

### CROSS-REFERENCE TO RELATED APPLICATION(S)

The present application derives priority from U.S. Provisional Patent Application 63/548,436 filed 14 Nov. 2023.

### BACKGROUND OF THE INVENTION

### 1. Field of the Invention

The present invention relates generally to prosthetics and more particularly, to a prosthetic leg mounting system for external ring fixators for faster post-operative ambulation and protection and enhanced healing of the residual limb (stump).

### 2. Description of the Background

When a patient encounters a traumatic injury to his or her lower limb, external ring fixators are often used as a method of immobilizing leg and other limb bones to allow a fracture to heal. An “external ring fixation assembly” for an amputee, sometimes called an “Ilizarov fixator” or “external ring fixator”, generally comprise one or more rings with radial pins or screws that are placed into the bone on all sides of the residual limb. The external fixator can be adjusted such that the bone remains in an optimal position during the healing process.

For example, U.S. Pat. No. 7,306,601 to McGrath et al. (Quantum Medical Concepts, Inc.) issued Dec. 11, 2007 shows a typical external ring fixation system.

It is also known that the healing process can be accelerated by getting the patient up and moving to increase circulation, which helps with healing. For this reason some patients have an additional ring, or footplate, attached beneath their foot that allows them to bear weight and ambulate. This additional ring or foot ring typically attaches with four (or more) threaded rods that are secured with multiple nuts above and below each ring.

For example, U.S. patent application No. 20150112339 by Lindahl et al. (Aalto University Foundation) published Apr. 23, 2015 shows an external ring fixator with attached shoe for controlling ankle movement.

U.S. Pat. No. 8,192,434 to Huebner et al. (Quantum Medical Concepts LLC) issued Jun. 5, 2012 shows an external ring fixation assembly with a foot-supporting plate.

U.S. Pat. No. 8,323,282 to Taylor issued Dec. 4, 2012 shows a walking plate for an orthopedic ring-fixator.

U.S. Pat. No. 9,381,129 to Vicik issued Jul. 5, 2016 (MGV Enterprises, Inc) shows an external ring-fixator and auxiliary support having a quick-release mechanism. For the very same reasons, it is likewise desirable to get amputates ambulating as soon as possible after their procedures, but this is more difficult and less common. This is noted by Lam et al where the authors attached a prosthetic leg to a weight-bearing external ring fixator to allow for early weight bearing and exercising. Lam et al, Lengthening of Tibia after Trans-Tibial Amputation: Use of a Weight Bearing External Fixator-Prosthesis Composite, HSS J. (Sep. 8, 2015). In this case, the prosthesis was simply screwed to a bottom ring.

In U.S. Pat. Nos. 10,485,679 and 11,185,428 issued Nov. 26, 2019 and Nov. 30, 2021, respectively, the present inventors disclose a prosthetic external ring fixation assembly that facilitates immediate attachment of a prosthetic leg and foot for immediate post-amputee ambulation to get the patient up and moving, increase circulation, expedite healing, and improve overall health and rehabilitation.

The present invention improves the concept with a monolithic support block that eases patient access, facilitates a broader range of configurations with fewer components, and generally improves wearability while simplifying manufacturing and reducing cost.

### SUMMARY OF THE INVENTION

In accordance with the foregoing it is an object of the invention to provide a prosthetic leg mounting system for external ring fixators that facilitates immediate attachment of a prosthetic leg and foot for immediate post-amputee ambulation to get the patient up and moving, increase circulation, and expedite healing.

The foregoing and other objects are accomplished with an improved prosthetic external ring fixation assembly for quick-connect adjustable attachment of a prosthetic leg for immediate post-amputee ambulation. The device is configured for use with an existing external fixation assembly for fixation to a femur or tibia, and an existing prosthetic leg and foot combination of choice. The assembly generally includes an articulating fixator adjustably-attached to a monolithic support block by locking-ball-joint struts, and quick-connect pins insertable through the fixator and into receptacles on the locking-ball-joint struts for releasable attachment. The monolithic support block is a disc or polyhedron configured on its underside as a mounting base for mounting the prosthetic leg/foot, and upwardly formed with one or more exposed faces defined by a plurality of receptacles for anchoring the struts. In an embodiment the monolithic support block has four raised weight-bearing turrets at each corner, each turret having an exposed face interrupted by a receptacle for anchoring a locking-ball-joint struts. In another embodiment the monolithic support block is a disc circumscribed by a beveled face having a plurality of radially-spaced receptacles for anchoring the locking-ball-joint struts. The quick-connect mechanism is a plurality of detent pins that allow releasable mounting of the fixator to the multi-faceted support block.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a perspective illustration of an external fixation assembly 2 for post-amputee prosthetics assembly according to the invention.

FIG. 2 is top perspective view of the external fixation assembly 2 of FIG. 1.

FIG. 3 is a close-up top perspective illustration of an embodiment of the multi-faceted support block 20 of FIGS. 1-2.

FIG. 4 is a close-up bottom perspective illustration of the multi-faceted support block 20 as in FIG. 3.

FIG. 5 is a perspective illustration of the multi-faceted support block of FIGS. 1-3 with receptacles 24 moved to the top face of each turret 22.

FIG. 6 is a perspective illustration of another embodiment the monolithic support block 120 shaped as a disc circumscribed by a beveled face 123 with a plurality of radially-spaced receptacles 124 for anchoring the locking-ball-joint struts.

FIG. 7 is a perspective illustration of an alternate embodiment of a monolithic support block 220 shaped as a tetrahedron with a plurality of exposed faces 223 each interrupted by a receptacle 224 for anchoring locking-ball-joint struts.

FIG. 8 is a perspective illustration of an alternate embodiment of a monolithic support block 320 shaped as a square with a singular upwardly-exposed face 323 interrupted by four corner receptacles 324 for anchoring locking-ball-joint struts.

### DETAILED DESCRIPTION OF THE PREFERRED EMBODIMENT

Reference will now be made in detail to preferred embodiments of the present invention, examples of which are illustrated in the accompanying drawings. Wherever possible, the same reference numbers will be used throughout the drawings to refer to the same or like parts.

The present invention is an improved fixation assembly for quick-connect adjustable attachment of a prosthetic leg post-amputee ambulation as soon as possible after the procedures.

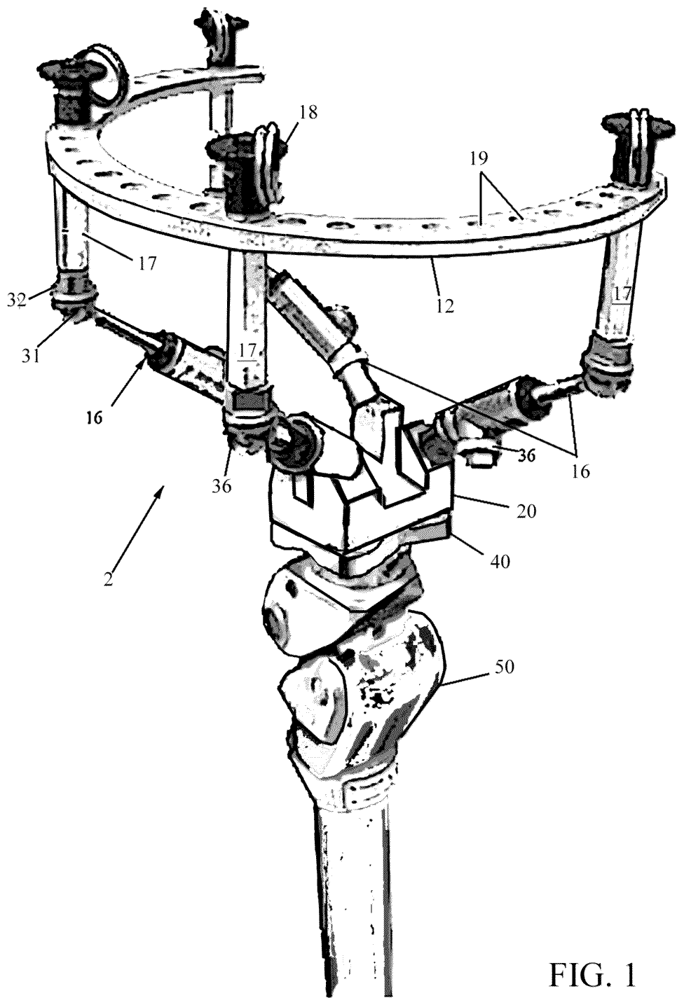

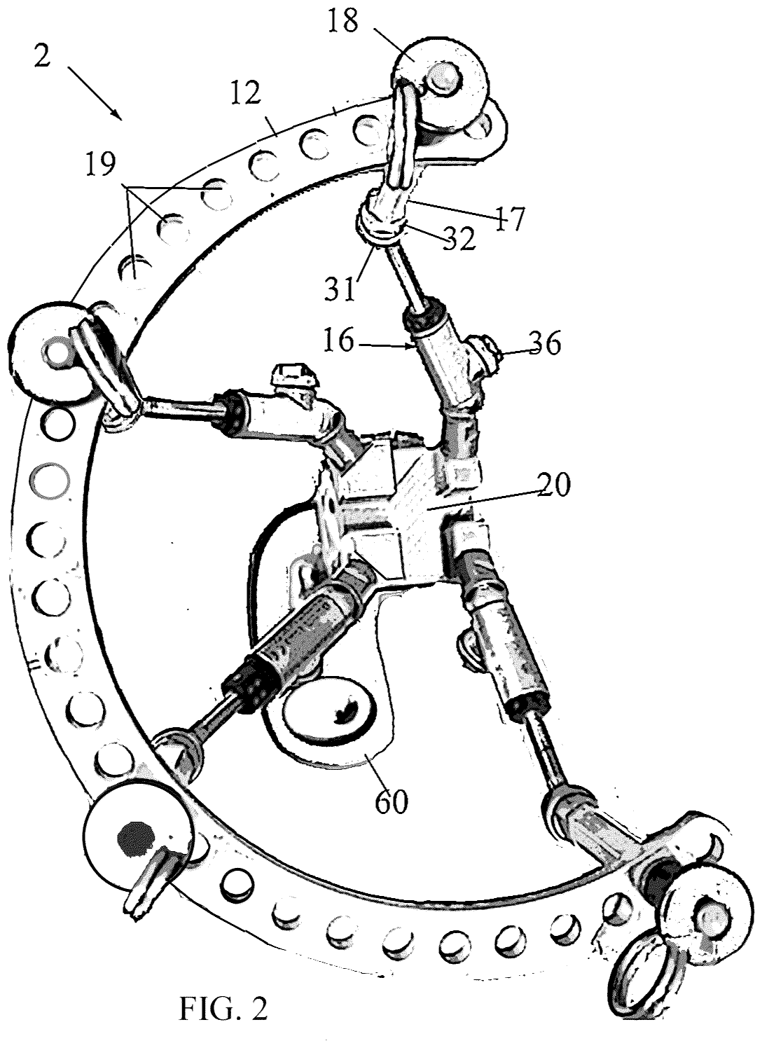

FIGS. 1-2 are perspective illustrations of the fixation assembly 2 for quick-release mounting of a prosthetic leg onto an existing fixation device according to the invention. The existing fixation device (not shown) is a conventional component used to stabilize bone and soft tissues a distance from an operative injury, such as a conventional external ring fixator attached to the thigh bone (femur) or to the tibia (shinbone). The present fixation assembly 2 generally includes a fixation bracket 12 adjustably-attached to a monolithic support block 20 by locking-ball-joint struts 16, and quick-connect pins 18 insertable through holes 19 in the fixation bracket 12 and into spacers 17 for quick-release attachment, spacers 17 in turn being threaded into receptacles 36 on the locking-ball-joint struts 16. The monolithic support block 20 is a multi-faceted weight-bearing block configured beneath as a mounting base for mounting the prosthetic leg/foot to the underside. The fixation bracket 12 may be a partial ring (as shown) or full ring having a plurality of radially-spaced holes 19. The quick-connect pins 18 are insertable through the holes 19 of fixation bracket 12 and into spacers 17 that are attached end-to-end to the locking-ball-joint struts 16. The struts 16 determine the spacing and/or angular disposition of the fixation bracket 12 and hold it in a substantially fixed relative disposition. The struts 16 may be arranged around the fixation bracket 12 as desired, preferably four struts 16 (and four spacers 17) at 0, 45, 90 and 135 degree equiangular increments, although three to six struts may be used as a matter of design choice. Each strut 16 preferably has a length-adjustment capability that allows the length of the strut 16 to be adjusted telescopically and then locked at length. Each strut 16 also preferably has an angular-adjustment capability via locking-ball-joint, thereby providing combination length and/or angular adjustment of the fixation bracket 12. The fixation bracket 12 may be connected to an existing external ring fixator or other conventional fixation device (not shown) in a conventional manner via any suitable pins, rods, and/or screws.

The plurality of spacers 17 are tubular members having a receptacle at one end configured to accept quick-connect pins 18 for removable securement to the fixation bracket 12. The quick-connect pins 18 are preferably detent lock pop-and-plunger pins, and most preferably button-handle lock pins, 2″ long and ¼″ diameter.

Each strut 16 is pivotally-connected to the opposing end of a corresponding spacer 17 by a screw-insert 32 threaded therein, the screw-insert 32 riding on a unidirectional pivot joint 31. Each strut 16 can be independently lengthened or shortened and fixed in length and/or orientation by a locking screw 36. The struts 16 are likewise secured by male screw-inserts threaded into the monolithic support block 20. The struts 16 may each be a Tru-Lock™ Rapid Strut manufactured by Orthofix SRL. This configuration allows for limited spatial adjustment (position, orientation and fixation) of the fixation bracket 12 (and external fixation ring assembly) relative to the monolithic support block 20 within a three-dimensional frame of reference.

The underside of monolithic support block 20 is configured with a prosthetic mounting plate 40. The mounting plate 40 may vary depending on the desired prosthetic. As an example, the mounting plate 40 may be a 4-hole pyramid adapter generally including a keyed male pin for docking in a conventional pyramid receptacle, the pin capable of being locked in position with set screws. The docking pin/receptacle combination further allows mounting a prosthetic leg 50 and foot 60 (FIG. 2) thereto, allowing for early weight bearing and exercising. The foot may be, for example, a foreshortened prostheses (“stubby”) such as shown and described in U.S. Pat. No. 9,301,859 to Haun, or any other suitable foot. An exemplary receptacle is found in in U.S. Pat. No. 8,252,066 to Haun.

The foregoing fixation assembly 2 is quickly attachable, fully adjustable, and allows the amputee patient to remain active and ambulatory with minimal assistive devices, improving circulation and healing as well as psychological advantages. The fixation bracket 12 also protects the residual stump post-amputation.

FIGS. 3-8 collectively illustrate several variations of the monolithic support block 20, 120, 220, 320, all being multi-faceted unitary weight-bearing members preferably formed of Kevlar™ or other lightweight yet strong fiber-reinforced plastic, or alternatively molded from, e.g., bisphenol A (BPA) plastic. Alternatively, the monolithic support blocks can be machined from billet aluminum or other suitable metal on a CNC machine to save cost.

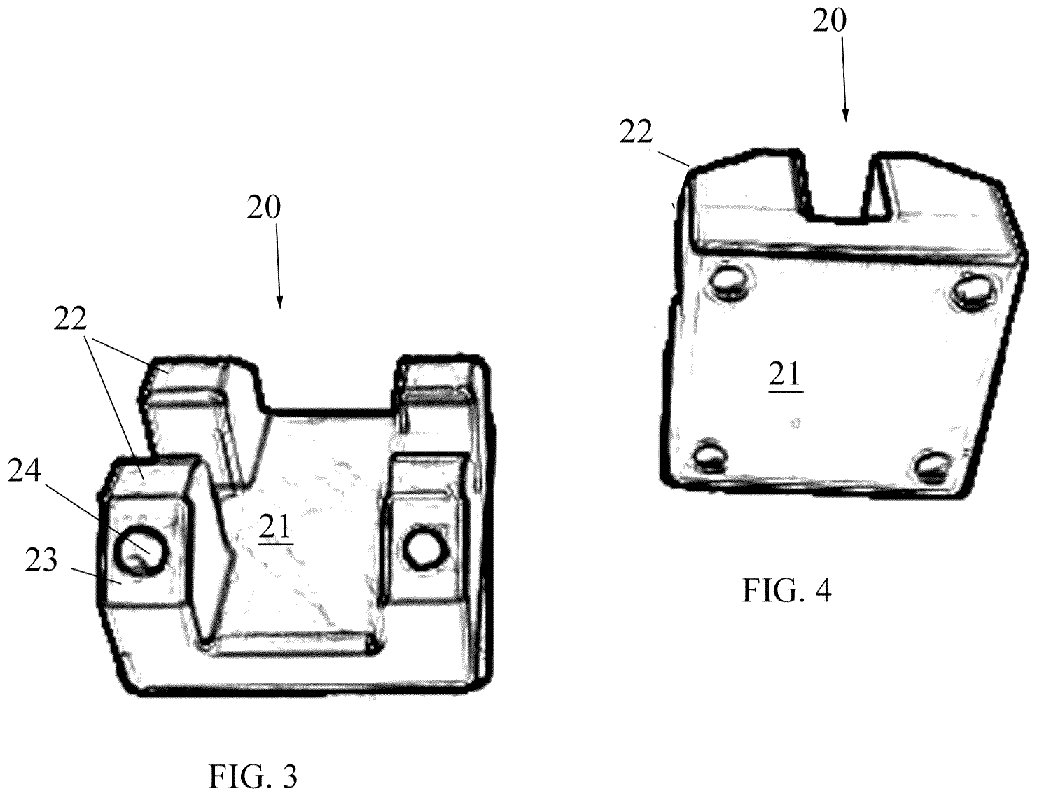

In the embodiment shown in FIGS. 3-4, the monolithic support block 20 is formed with a flat base 21 having an underside (FIG. 4) configured for coupling any standard prosthetic mounting plate 40 for attaching prosthetic limbs or feet (e.g., pyramid adapters). As seen in FIG. 3 the topside of monolithic support block 20 is formed with four raised turrets 22 protruding upward from base 21. Each turret 22 preferably comprises a polyhedron protruding upward from base 21 and configured with an outwardly-facing beveled face 23 for angular support of the locking-ball-joint struts 16. A threaded receptacle 24 enters the center of each beveled face 23 perpendicular for maximum angular support of the locking-ball-joint struts 16, which are secured by male screw-inserts threaded into the receptacles 24 of monolithic support block 20. The beveled faces 23 each provide a weight-bearing support surface for a corresponding strut 16, and weight-bearing capacity is maximized when the strut 16 protrudes perpendicular to the beveled face 23, outward and upward from the prosthetic mounting plate 40. The turrets 22 may be arranged at various angles as a matter of design choice to accommodate different fixation ring types and improve block 20-to-fixation bracket 12 alignment of the struts 16. For example, the beveled faces 23 of one pair of turrets 22 may be co-planar and face diametrically away from the co-planar beveled faces 23 of the other pair of turrets 22 as seen in FIG. 3. Alternatively, the beveled faces 23 of each of the four turrets 22 may be radially oriented away from each other, for example at 90 degree angles. As still another alternative, the beveled faces 23 of one pair of turrets 22 may be face diametrically away from each other as seen in FIG. 2, while the beveled faces 23 of the other pair of turrets 22 are radially oriented away from the first pair and from each other at 90 degree angles.

The distance of the monolithic support block 20 from the fixation bracket 12 and/or the offset angle of the prosthetic foot may be adjusted by adjusting the length and/or pivot angles of the unidirectional-pivoting length adjustable struts 16. After use, the entire external fixation assembly 2 may be quick-released and removed by removing the quick-connect pins 18.

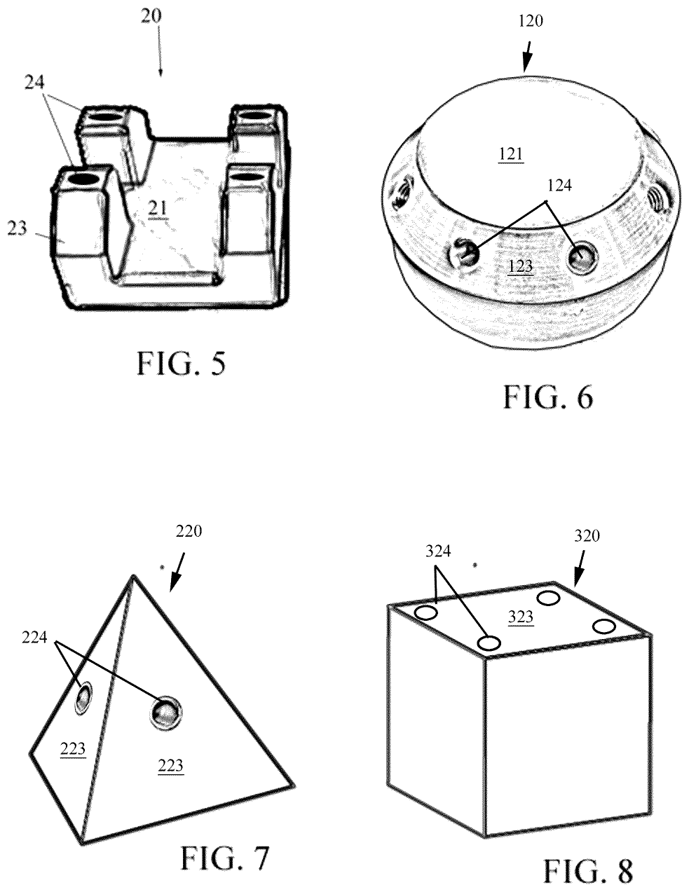

One skilled in the art should understand that the monolithic support block 20 may take a variety of shapes and configurations all capable of providing a plurality of receptacles in weight-bearing surface(s) for anchoring the struts 16. For example, FIG. 5 is a perspective illustration of the monolithic support block 20 as in FIGS. 1-3 but with receptacles 24 moved to the top of each turret 22.

FIG. 6 is a perspective illustration of another embodiment the monolithic support block 120 shaped as a puck or disc circumscribed by a beveled face 123 and having a plurality of radially-spaced receptacles 124 formed along the beveled face 123 for anchoring the locking-ball-joint struts 16.

FIG. 7 is a perspective illustration of an alternate embodiment of a monolithic support block 220 shaped as a tetrahedron with a plurality of exposed angled faces 223 each interrupted by a receptacle 224 for anchoring locking-ball-joint struts.

FIG. 8 is a perspective illustration of an alternate embodiment of a monolithic support block 320 shaped as a square with a singular upwardly-exposed face 323 interrupted by four corner receptacles 324 for anchoring locking-ball-joint struts.

It should now be apparent that the foregoing external fixation assembly 2 provides quick-connect adjustable attachment of a prosthetic leg for immediate post-amputee ambulation. The device is quickly attachable, fully adjustable, and allows the amputee patient to remain active and ambulatory almost immediately with minimal assistive devices, improving circulation and healing as well as psychological advantages.

Those skilled in the art will understand that various modifications and variations can be made in the present invention without departing from the spirit or scope of the invention. It is to be understood, therefore, that the invention may be practiced otherwise than as specifically set forth in the appended claims.

What is claimed is:

1. An apparatus for attachment of a prosthetic leg to an external fixation assembly, comprising:
a fixation bracket;
a monolithic support block configured for attachment atop a prosthesis and protruding upward to at least one exposed face;
a plurality of struts each attached at one end to said at least one exposed face and attached at another end to said fixation bracket.

2. The apparatus according to claim 1, wherein said plurality of struts are each length-adjustable and pivotally attached to the fixation bracket.

3. The apparatus according to claim 2, wherein each of said plurality of length-adjustable struts includes a lock for fixing length.

4. The apparatus according to claim 1, wherein said struts each comprise a quick-release coupling mechanism.

5. The apparatus according to claim 4, wherein said struts each comprise a uni-directional ball joint.

6. The apparatus according to claim 1, wherein said fixation bracket is semi-circular and said plurality of holes are radially spaced.

7. The apparatus according to claim 1, wherein said monolithic support block comprises a disc having one upwardly exposed face.

8. The apparatus according to claim 1, wherein said monolithic support block comprises a polyhedron.

9. The apparatus according to claim 1, wherein said monolithic support block comprises a plurality of raised turrets protruding upward and bearing an outwardly-exposed beveled face.

10. The apparatus according to claim 9, wherein said monolithic support block comprises four raised turrets.

11. The apparatus according to claim 9, wherein each said turret is defined by a threaded screw-hole.

12. An apparatus for attachment of a prosthetic leg to an external fixation assembly, comprising:
a fixation bracket having a plurality of spaced holes;
a monolithic support block formed with a base having an underside configured for connection of a prosthesis and a topside formed with a plurality of outwardly-exposed faces;
a plurality of struts each attached at one end to the beveled face of a corresponding turret of the support block and releasably-attached at another end into a hole of the fixation bracket.

13. The apparatus according to claim 12, wherein said plurality of struts are each length-adjustable and pivotally attached to the fixation bracket.

14. The apparatus according to claim 12, wherein said struts each comprise a quick-release coupling mechanism.

15. The apparatus according to claim 12, wherein said struts each comprise a uni-directional ball joint.

16. The apparatus according to claim 12, wherein said monolithic support block comprises one of a disc or polyhedron.

17. The apparatus according to claim 1, wherein said monolithic support block comprises a plurality of raised turrets protruding upward, each said turret bearing one of said plurality of exposed faces.

18. The apparatus according to claim 17, wherein the face of each of said turrets is interrupted by a threaded screw-hole.

19. A monolithic support block for attachment of a prosthesis, comprising a base having an underside configured for connection of said prosthesis and an upside formed with at least one outwardly-exposed face with at least one receptacle formed therein for providing a weight-bearing support surface for a strut inserted into said receptacle.

20. The apparatus according to claim 19, wherein said monolithic support block comprises a plurality of raised turrets protruding upward from said base, each of said plurality of turrets having an outwardly-exposed face with a receptacle formed therein.
